(19) United States
(12) Patent Application Publication
(10) Pub. No.: US 2026/0137195 A1
(43) Pub. Date: May 21, 2026

(54) METHODS OF CONTROLLING THE APPEARANCE OF A LIP PRODUCT

(71) Applicant: ELC MANAGEMENT LLC, Melville, NY (US)

(72) Inventor: Christopher Aidan, Austin, TX (US)

(21) Appl. No.: 18/954,664

(22) Filed: Nov. 21, 2024

(51) Int. Cl. A45D 44/00; A45D 40/00; A45D 40/24

(52) U.S. Cl. CPC A45D 44/00; A45D 40/24; A45D 2040/0006

(57) ABSTRACT

A method of controlling an appearance of a lip product includes maintaining a mobile application in a mobile device memory included in a mobile device, and the mobile application includes a color selection feature containing a plurality of shade options and a plurality of intensity options. The method includes selecting a shade selection from the plurality of shade options and an intensity selection from the plurality of intensity options. The method further includes communicating, the shade selection and the intensity selection to a color-changing lipstick device.

### FIELD OF THE DISCLOSURE

The present disclosure generally relates to a color-changing cosmetic and cosmetic application device. More specifically, the present disclosure relates to a lipstick product containing an electrochromic material and a device for providing electrical current to the lipstick product to cause a color change.

### BACKGROUND

A traditional cosmetic, such as lipstick, contains a pigment that gives the cosmetic a specific color. Wearers often own the same cosmetic in multiple colors in order to vary the appearance of their makeup for different events, seasons, and styles. Owning multiple colors of a cosmetic is burdensome. Storing multiple cosmetics in an organized manner is time and space consuming. Even if a wearer only wishes to wear a particular color once, the wearer is often forced to purchase a full-sized version of the cosmetic that has far more product than required. If a wearer uses multiple colors of a cosmetic in a single look (e.g., a first color to outline the lips and another for the interior portion of the lips), the wearer must carry two different cosmetics if she wishes to touch up the appearance of her makeup later.

### SUMMARY

Examples within the scope of the present disclosure are directed to a color-changing lipstick product, a device for the color-changing lipstick product, and methods of use for the color-changing lipstick product.

In an example, a method of controlling an appearance of a lip product includes maintaining a mobile application in a mobile device memory included in a mobile device. The mobile device includes a mobile device display, a mobile device interface, and a controller comprising a non-transitory memory and one or more processors. The mobile application includes a color selection feature containing a plurality of shade options and a plurality of intensity options. The method further includes selecting, using the one or more processors and the mobile device interface of the mobile device, a shade selection from the plurality of shade options and an intensity selection from the plurality of intensity options. The method further includes communicating, using the one or more processors of the mobile device, the shade selection and the intensity selection to a color-changing lipstick device.

In an approach, the method may further include uploading, using the one or more processors of the mobile device, a photograph to the mobile application. The method may further include identifying, using the one or more processors of the mobile device, a suggested shade option from the plurality of shade options and a suggested intensity option from the plurality of intensity options based upon the photograph. The method may further include displaying, using the one or more processors of the mobile device and the mobile device display, the suggested shade option and the suggested intensity option.

In an approach, the method may include displaying, using the one or more processors of the mobile device and the mobile device display, a combined example of the suggested shade option and the suggested intensity option.

In an approach, the method may include selecting or declining, using the one or more processors of the mobile device and the mobile device interface, the suggested shade option and the suggested intensity option.

In an approach, the method may include sending, using the one or more processors of the mobile device, a request for GPS data to the color-changing lipstick device.

In an approach, the method may include receiving, via a communication module of the color-changing lipstick device and one or more processors of the color-changing lipstick device, the shade selection and the intensity selection, and adjusting, using one or more processors of the color-changing lipstick device, an electrical current provided to a color-changing lip product of the color-changing lipstick device based on the shade selection and the intensity selection.

In an approach, the method may include receiving, using the one or more processors of the mobile device, sensor data collected by a sensor of the color-changing lipstick device. The method may further include activating, using the one or more processors, an alert of the mobile device.

In an approach, the alert may include displaying, using the one or more processors of the mobile device, the sensor data on the mobile device display.

In an approach, the alert may including vibrating, using the one or more processors of the mobile device, the mobile device.

In an approach, the alert may include generating, using the one or more processors of the mobile device, a sound from the mobile device.

In an approach, the method may include providing, using the one or more processors of the mobile device, a plurality of input options based on the sensor data. The method may include selecting, using the one or more processors of the mobile device and the mobile device interface, one or more of the plurality of input options. The method may include communicating, via the one or more processors of the mobile device, the selected one or more of the plurality of input options to the color-changing lipstick device.

In an approach, the sensor data may include sensor data from at least one of a temperature sensor, a GPS sensor, a UV sensor, a battery sensor, a light sensor, and a motion sensor.

In an approach, the input options may include adjusting an electrical current provided to a lip product of the color-changing lipstick device, turning on a fan of the color-changing lipstick device, identifying a location of the color-changing lipstick device, beginning a low-power mode of operation of the color-changing lipstick device, selecting a new shade selection from the plurality of shade options, and selecting a new intensity selection from the plurality of intensity options.

In an approach, the method may further include receiving, via a communication module of the color-changing lipstick device and the one or more processors of the color-changing lipstick device, the selected one or more of the plurality of input options and implementing the selected one or more of the plurality of input options.

In an approach, the selected one or more of the plurality of input options may include at least one of adjusting an electrical current provided to the lip product of the color-changing lipstick device, selecting a new shade selection from the plurality of shade options, and selecting a new intensity selection from the plurality of intensity options, and implementing the selected one or more of the plurality of input options may include controlling a power source, using the one or more processors of the color-changing lipstick device, to adjust the electrical current provided to the lip product.

In an approach, the selected one or more of the plurality of input options may include turning on a fan of the color-changing lipstick device, and implementing the selected one or more of the plurality of input options may include controlling the fan, using the one or more processors of the color-changing lipstick device.

In an approach, the selected one or more of the plurality of input options may include identifying a location of the color-changing lipstick device, and implementing the selected one or more of the plurality of input options may include activating, using the one or more processors of the color-changing lipstick device, a GPS sensor to collect sensor data regarding a location of the color-changing lipstick device, and communicating, using the communication module of the color-changing lipstick device and the one or more processors of the color-changing lipstick device, the sensor data regarding the location of the color-changing lipstick device to the mobile device.

In an approach, the selected one or more of the plurality of input options may include beginning a low-power mode of operation of the color-changing lipstick device, and implementing the selected one or more of the plurality of input options may include controlling a power source, using the one or more processors of the color-changing lipstick device, to reduce usage of the power source.

In an approach, the method may further include displaying, using the one or more processors of the mobile device and the mobile device display, color examples in conjunction with the plurality of shade options.

In an approach, the method may further include displaying, using the one or more processors of the mobile device and the mobile device display, intensity examples in conjunction with the plurality of intensity options.

In an approach, the method may further include displaying, using the one or more processors of the mobile device and the mobile device display, a combined example of the shade selection and the intensity selection.

In an approach, the method may further include requesting, using the one or more processors of the mobile device and the mobile device interface, confirmation of the shade selection and the intensity selection after displaying the combined example and before communicating the shade selection and the intensity selection to the color-changing lipstick device.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various examples. Also, common but well-understood elements that are useful or necessary in a commercially feasible examples are often not depicted to facilitate a less obstructed view of these various examples. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### BRIEF DESCRIPTION OF THE DRAWINGS

The figures described below depict various aspects of the system and methods disclosed herein. It should be understood that each figure depicts an arrangement of a particular aspect of the disclosed system and methods, and that each of the figures is intended to accord with a possible arrangement thereof.

There are shown in the drawings arrangements which are presently discussed, it being understood, however, that the present embodiments are not limited to the precise arrangements and instrumentalities shown, wherein:

FIG. 1 illustrates a perspective view of a color-changing lipstick device including a lip product comprising an electrochromic material, a case, and a mobile device in communication with the color-changing lipstick device.

FIG. 2 illustrates a cross-sectional view of the color-changing lipstick device of FIG. 1.

FIG. 3 illustrates a cross-sectional view of a color-changing lipstick device including a roller.

FIG. 4 illustrates schematically a controller of the color-changing lipstick device of FIGS. 1-4.

FIG. 5 illustrates schematically a method of controlling an appearance of a lip product.

FIG. 6 illustrates a perspective view of a mobile device with a mobile application to control a color-changing lipstick device, the mobile application displaying a plurality of shade options and a plurality of intensity options.

FIG. 7 illustrates a perspective view of the mobile application of FIG. 6 displaying a photograph-matching feature.

FIG. 8 illustrates a perspective view of the mobile application of FIGS. 6 and 7 displaying a plurality of input options based on sensor data.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various examples. Also, common but well-understood elements that are useful or necessary in a commercially feasible examples are often not depicted to facilitate a less obstructed view of these various examples. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various approaches, a color-changing lipstick product is provided that can change colors when an electric current is provided by a color-changing lipstick device. The color-changing lipstick product can be set to achieve a number of different shades and intensities, thereby giving a wearer a variety of options. The color-changing lipstick product and device reduces or eliminates the need to own multiple lipsticks in different colors and simplifies the process of bringing along makeup for a later touch-up. Further, the color-changing lipstick device can be used in combination with a mobile application that displays shade and intensity examples prior to their selection by a user to simplify the process of changing the color-changing lipstick product to a desired color. The mobile application may also include a photo-feature that makes color recommendations based upon an uploaded photograph. This simplifies the process of color selection and facilitates a satisfactory cosmetic application by a wearer.

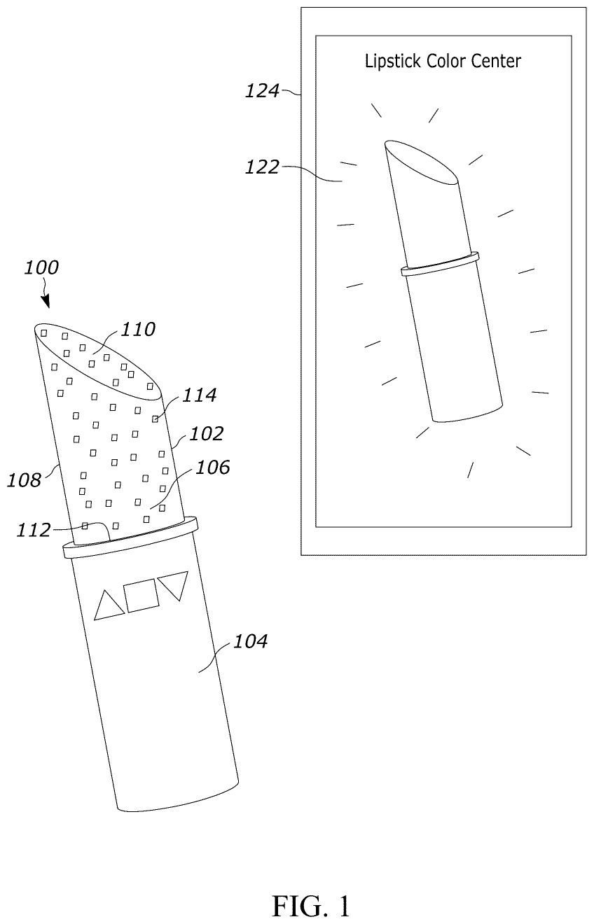

FIG. 1 shows a color-changing lipstick device 100 that includes a color-changing lip product 102 and a case 104. The color-changing lip product 102 includes a base substance 106. For example, the base substance 106 may be a blend of various waxes, oils, and butters. For example, beeswax may be an ingredient in the base substance 106 to provide structure and stability to the base substance 106 prior to application and to provide a protective barrier to lock in moisture once applied to a wearer's lips. Alternately, a plant-based wax such as a candelilla wax, carnauba wax, or soy wax may be used to create a vegan product. The base substance 106 may include emollient oils, such as coconut oil, jojoba oil, almond oil, and avocado oil, to imbue the base substance 106 with moisturizing properties that can add hydration to a wearer's lips and can also making spreading the base substance 106 easier. The base substance 106 may further include lanolin to moisturize dry lips and antioxidants and vitamins, such as vitamin E or plant extracts. In some forms, the color-changing lip product 102 may be a liquid with, for example, a base substance 106 that is an oil such as mineral oil, castor oil, or another lightweight oil.

In some forms, the base substance 106 may be shaped into a three-dimensional form 108. The three-dimensional form 108 includes an application surface 110 for applying the lip product 102 to a wearer and a connection surface 112 for securing the lip product 102 within the case 104. The application surface 110 may, for example, be disposed at an angle relative adjacent surfaces to allow ergonomic application of the lip product 102. The connection surface 112 may, for example, include divots or slots to facilitate the connection to the case 104. In other arrangements, as discussed further below, a liquid lip product 102 may be applied by the color-changing lipstick device 100 without the lip product 102 forming a three-dimensional form 108.

An electrochromic material 114 is distributed through the base substance 106. The electrochromic material is configured to change color in response to application of an electrical current. Example electrochromic materials include polymers such as Poly(3,4-ethylenedioxythiophene) (PEDOT) and Poly(3,4-ethylenedioxythiophene):poly(styrenesulfonate) (PEDOT: PSS), metal oxides such as tungsten oxide and nickel oxide, conducting polymers such as polyaniline and polythiophene, carbon-based materials such as graphene and graphene oxide, and organic molecules such a viologens and ruthenium complexes. More than one electrochromic material 114 may be distributed through the base substance 106. The choice of which electrochromic material 114 to include in the lip product 102 may be based on how closely a desired color change corresponds to the color change experiences by an electrochromic material 114 when an electrical current is applied. Other considerations include biocompatibility, cost, and interactions with other ingredients of the lip product 102.

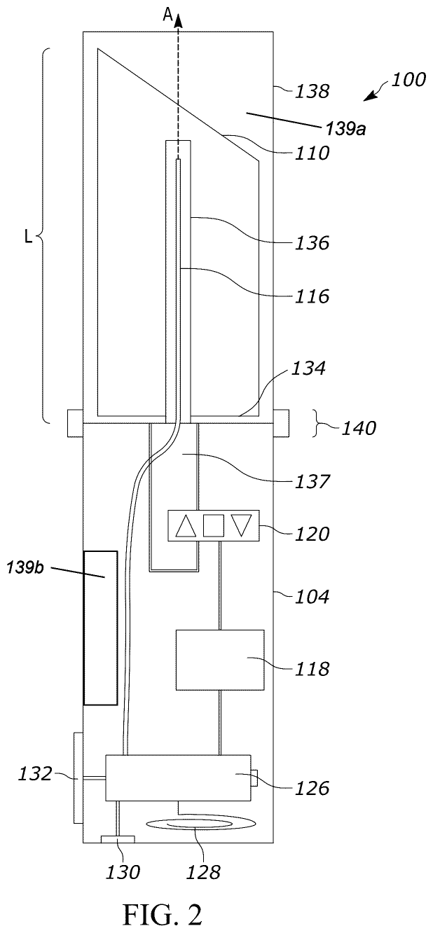

As shown in FIG. 2, the case 104 includes a power source 116 in electrical communication with the lip product 102 and a controller 118 in communication with the power source 116. A user interface 120 is in communication with the controller. The user interface 120 may be partially or fully integrated into the case 104. In the arrangement shown, the user interface 120 is a plurality of buttons (e.g., a + and − button and an on/off button to control the state and amount of electrical current provided to the lip product 102). In other arrangements, the user interface 120 may include other features such as an integrated display, an interactive touch screen, and/or LED lights. A user of the color-changing lipstick device 100 can provide input via the user interface 120, which is provided to the controller 118, which in turn controls the power source 116 to provide electrical current to the lip product 102 to cause the lip product 102 to change color. In some arrangements, as shown in FIG. 1, the color-changing lipstick device 100 may include a separate display 122 in communication with the controller 118 and positioned in or integrated into, for example, a mobile device 124.

The power source 116 shown in FIG. 2 may include a rechargeable battery 126. The rechargeable battery 126 may be charged by a connection to an external power source. The power source 116 may include a wireless charging coil 128 to enable wireless charging of the rechargeable battery 126. Instead, or in addition, the case 104 may include a port 130 for connecting the power source 116 to an external power outlet by a charging cord. Further, in some arrangements, the power source 116 may include solar panels 132 and excess solar power may be stored in the rechargeable battery 126.

In the arrangement shown in FIG. 2, the case 104 includes a mounting structure 134 that includes the power source 116. The mounting structure 134 may include, for example, projections or textured surfaces to secure the lip product 102 in place. In the arrangement shown, the lip product 102 is substantially cylindrical and arranged along an axis A. The mounting structure 134 includes a projection 136 that extends partially along the axis A. In some arrangements, the projection 136 may extend along a majority of a length L of the lip product 102, stopping short of the application surface 110 to prevent direct contact with a wearer's lips during application of the lip product 102. The projection 136 may be retractable into a cavity 137 so that it can support the lip product 102 initially and retract as the lip product 102 is used up to avoid contact with a wearer's lips. In addition to physically keeping the lip product 102 connected to the case 104, the mounting structure 134 may deliver electrical current from the power source 116. The electrical current may be provided uniformly from the power source 116 (e.g., along the projection 136) so that the lip product 102 is all changed to a uniform color. Alternately, the electrical current may be provided at a gradient along the axis A to cause the lip product 102 to have an ombre appearance (i.e., to transition between shades of a color along the length of the axis A).

A lid 138 covers the three-dimensional form 108 of the lip product 102. The lid 138 is secured around the lip product 102 and around the mounting structure 134. A lock 140 secures the lid 138 around the lip product 102. The lock 140 may be a threaded connection, a snap-fit, a magnetic closure, or the like. The lid 138 and the lock 140 not only keep the lip product 102 hygienic for application on a wearer's lips but also prevent inadvertent transmission of the electrical current from the power source 116 to anything other than the lip product 102. In some arrangements, the lid may form a first compartment 139a containing a first cosmetic product (e.g., the lip product 102), and the case 104 may further include a second compartment 139b containing an auxiliary cosmetic product (e.g., a balm or high shine gloss to layer on the lip product 102 or a non-lip cosmetic such as eye shadow).

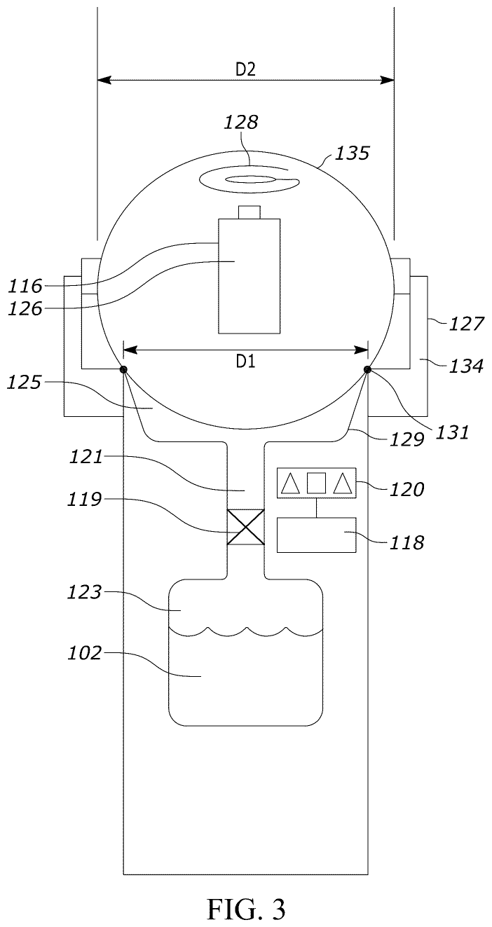

FIG. 3 illustrates an alternate case 104 for use with a lip product 102 that is a liquid illustrating an alternate mounting structure 134. Features of the case 104 in FIG. 2 may be implemented in FIG. 3 and vice versa. In the arrangement shown in FIG. 3, the case 104 includes a product cavity 129 having a cavity opening 131. The product cavity 129 is configured to contain the lip product 102, preferably in liquid form. The mounting structure 134 includes a roller 135. The mounting structure 134 is positioned at the cavity opening 131, and the roller 135 is in electrical communication with the power source 116 and is configured to provide electrical current to the lip product 102 to cause the lip product 102 to change color. The cavity opening 131 has a cavity diameter D1 and the roller 135 has a roller diameter D2 that is greater than the cavity diameter D1. As a result, the roller 135 can be rotatably secured against the cavity opening 131 by a mounting bracket 127. In other arrangements, the roller 135 may have a roller diameter D2 that is less than the cavity diameter D1, and the roller 135 may be secured within the product cavity 129 with walls of the product cavity 129 functioning as the mounting bracket 127.

The product cavity 129 includes an application chamber 125 adjacent the cavity opening 131 and a storage chamber 123 connected to the application chamber 125 by a product pathway 121. Liquid lip product 102 is stored within the storage chamber 123 and travels, as a result of movement of the color-changing lipstick device 100, to the application chamber 125. In the arrangement shown, the product pathway includes a one-way valve 119 along the product pathway 121 such that lip product flows only from the storage chamber 123 to the application chamber 125. The storage chamber 123 has a greater volume than the application chamber 125 as the application chamber 125 is optimally sized to contain only enough volume of lip product 102 for a single application. The roller 135 is in wireless communication with the controller 118, which is in turn in communication with the user interface 120, and the power source 116 is positioned within the roller 135. Electrical current provided by the roller 135 in response to instructions received from the controller 118 causes the lip product 102 on the roller 135 and/or in the application chamber 125 to change colors. In other arrangements not depicted herein, the product cavity 129 may have only a single chamber, and the lip product 102 may change color only when in direct contact with the roller 135.

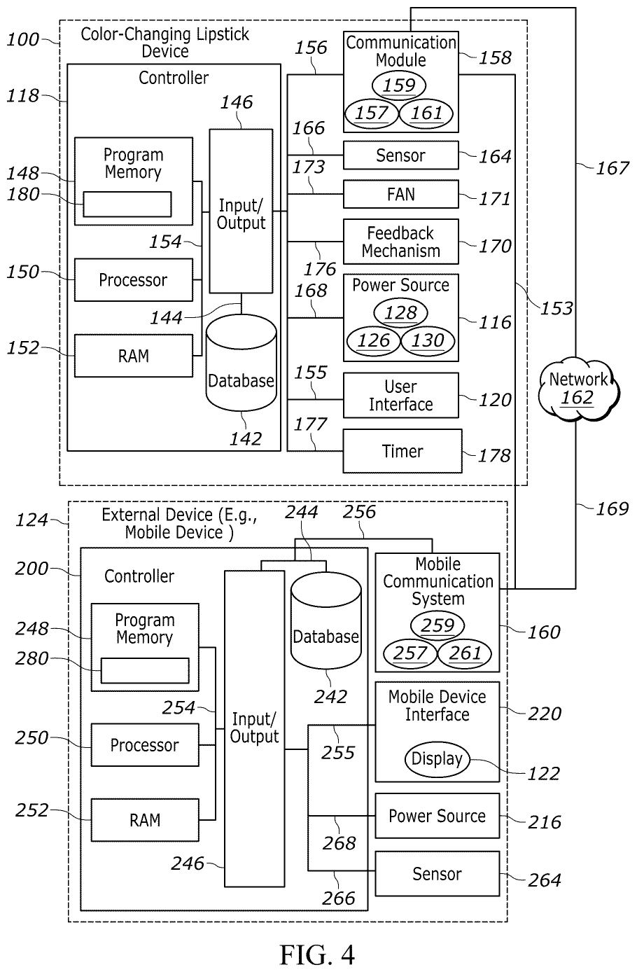

FIG. 4 illustrates schematically the controller 118. The controller 118 is configured to execute the functions of the disclosed arrangements in order to control operation of the color-changing lipstick device 100. The controller 118 may be operatively connected to a database 142 via a link 144 connected to an input/output (I/O) circuit 146. The database 142 may store, for example, a plurality of shade options associated with respective specific electrical currents, a plurality of intensity options associated with respective specific electrical currents, a plurality of activation options associated with an on/off status of an electrical current, and/or sensor data. It should be noted that, while not shown, additional databases may be linked to the controller 118 in a known manner. The controller 118 includes a program memory 148, one or more processors 150 (may be called microcontrollers or a microprocessors), a random-access memory (RAM) 152, and the input/output (I/O) circuit 146, all of which are interconnected via an address/data bus 154. It should be appreciated that although only one processor 150 is shown, the controller 118 may include multiple microprocessors 150. Similarly, the controller 118 may include multiple RAMs 152 and multiple program memories 148 Although the I/O circuit 146 is shown as a single block, it should be appreciated that the I/O circuit 146 may include a number of different types of I/O circuits. The RAM(s) 152 and the program memories 148 may be implemented as semiconductor memories, magnetically readable memories, and/or optically readable memories, for example.

A link 155, which may include one or more wired and/or wireless (Bluetooth, WLAN, etc.) connections, may operatively connect the controller 118 to the user interface 120 through the I/O circuit 146. A link 156, which may include one or more wired and/or wireless (Bluetooth, WLAN, etc.) connections, operatively connects the controller 118 to a communication module 158 of the color-changing lipstick device 100. The communication module 158 may communicate with a mobile communication system 160 of the mobile device 124 (an exemplary external device) via a network 162. The communication module includes at least one of a Bluetooth chip 157, an NFC chip 159, and an RFID chip 161. Further, one or more sensors 164 to collect sensor data is operatively connected to the controller 118 through the I/O circuit 146 by a link 166. For example, a sensor 164 may be a GPS sensor configured to collect location data, a temperature sensor to collect temperature data, a UV sensor to collect UV data, a battery sensor to collect battery life data, a light sensor to collect ambient light data, or a motion sensor to detect movement. Further, the power source 116 (including, for example, rechargeable battery 126, wireless charging coil 128, and port 130) is connected to the controller 118 by a link 168. In addition, the case 104 may include a feedback mechanism 170 to alert a user. The feedback mechanism 170 may be a haptic feedback mechanism (e.g., the feedback mechanism may include a motor 172 to create vibrations) or an auditory feedback mechanism (e.g., the feedback mechanism may include a speaker 174 to produce a sound). In some implementations the display 122 of the mobile device 124 may function as the feedback mechanism 170 by providing a visual alert. In the arrangement shown, the feedback mechanism 170 is connected to the controller 118 by a link 176. A link 177 may further connect the controller 118 to a timer 178. A link 173 may connect the controller 118 to a fan 173, which may be used to control the temperature of the lip product 102 or areas of the color-changing lipstick device 100. Other links are contemplated as within the scope of this disclosure.

The program memory 148 and/or the RAM 152 may store various applications (i.e., machine readable instructions) for execution by the processor 150. The program memory 148 and/or the RAM 152 may also store a variety of subroutines 180 for accessing specific functions of the controller 118. By way of example, and without limitation, the subroutines 180 may include, among other things:
a subroutine for providing, using the one or more processors 150 and user interface 120 and/or display 122, a plurality of shade options;
a subroutine for providing, using the one or more processors 150 and user interface 120 and/or display 122, a plurality of intensity options;
a subroutine for providing, using the one or more processors 150 and a user interface 120 and/or display 122, a plurality of activation options, wherein the activation options may be limited to an on or off status of an electrical current;
a subroutine for adjusting, using the one or more processors 150, an electrical current in response to a shade selection from one of a plurality of shade options;
a subroutine for adjusting, using the one or more processors 150, an electrical current in response to an intensity selection from one of a plurality of intensity options;
a subroutine for adjusting, using the one or more processors 150, an electrical current in response to an activation selection from one of the plurality of activation options, wherein the adjustment may be an on/off adjustment of the electrical current;
a subroutine for sending, using the one or more processors 150 and communication module 158, location data to an external device, such as mobile device 124;
a subroutine to, using the one or more processors 150, illuminate display 122 to reflect the sensor data from a sensor 164;
a subroutine to, using the one or more processors 150, adjust the electrical current provided to the lip product 102 in response to ambient light data;
a subroutine to, using the one or more processors 150, activate the feedback mechanism 170 in response to sensor data when the sensor data is beyond a preset threshold, for example, sounding an auditory alarm via speaker 174 if a temperature sensed by a sensor 164 within the case 104 exceeds a maximum desirable temperature;
a subroutine to, using the one or more processors 150, distribute electrical current to the lip product 102 based on motion data from a sensor 164 that is a motion sensor;
a subroutine to, using the one or more processors 150, adjust the electrical current based on the timer 178, for example, to turn off the electrical current after a pre-set amount of time has passed according to the time since a last instance of the electrical current being turned on.

FIG. 4 includes a mobile device 124, which may be in communication with the color-changing lipstick device 100 directly via a link 153 between the communication module 158 of the color-changing lipstick device 100 and the mobile communication system 160 of the mobile device 124 or indirectly via respective links 167 and 169 connecting the communication module 158 and the mobile communication system 160 to network 162. The mobile device 124 includes any or all of the components common to smartphones with select components identified below. The mobile device 124 includes a controller 200. The controller 200 may be operatively connected to a database 242 via a link 244 connected to an input/output (I/O) circuit 246. Like database 142, the database 242 may store, for example, a plurality of shade options associated with respective specific electrical currents, a plurality of intensity options associated with respective specific electrical currents, a plurality of activation options associated with an on/off status of an electrical current, and/or sensor data.

It should be noted that, while not shown, additional databases may be linked to the controller 200 in a known manner. The controller 200 includes a program memory 248, one or more processors 250 (may be called microcontrollers or a microprocessors), a random-access memory (RAM) 252, and the input/output (I/O) circuit 246, all of which are interconnected via an address/data bus 254. It should be appreciated that although only one processor 250 is shown, the controller 200 may include multiple microprocessors 250. Similarly, the controller 200 may include multiple RAMs 252 and multiple program memories 248 Although the I/O circuit 246 is shown as a single block, it should be appreciated that the I/O circuit 246 may include a number of different types of I/O circuits. The RAM(s) 252 and the program memories 248 may be implemented as semiconductor memories, magnetically readable memories, and/or optically readable memories, for example.

A link 255, which may include one or more wired and/or wireless (Bluetooth, WLAN, etc.) connections, may operatively connect the controller 200 to a mobile device interface 220, which may include the display 122, through the I/O circuit 246. A link 256, which may include one or more wired and/or wireless (Bluetooth, WLAN, etc.) connections, operatively connects the controller 200 to the mobile communication system 160. The communication module 158 may communicate with a mobile communication system 160 of the mobile device 124 (an exemplary external device) via a network 162. The mobile communication system 160 includes at least one of a Bluetooth chip 257, an NFC chip 259, and an RFID chip 261. Further, one or more sensors 264 to collect sensor data is operatively connected to the controller 200 through the I/O circuit 246 by a link 266. A power source 216 is connected to the controller 200 by a link 268. Other links are contemplated as within the scope of this disclosure.

The program memory 248 and/or the RAM 252 may store various applications (i.e., machine readable instructions) for execution by the processor 150. The program memory 248 and/or the RAM 252 may also store a variety of subroutines 280 for accessing specific functions of the controller 200. By way of example, and without limitation, the subroutines 280 may include, among other things:
a subroutine for selecting, using the one or more processors 250 and the mobile device interface 220, a shade selection from the plurality of shade options and an intensity selection from the plurality of intensity options;
a subroutine for communicating, using the one or more processors 250, the shade selection and the intensity selection to a color-changing lipstick device 100;
a subroutine for displaying, using the one or more processors 250 and the mobile device display 122, color examples in conjunction with the plurality of shade options and intensity examples in conjunction with the plurality of intensity options;
a subroutine for displaying, using the one or more processors 250 and the mobile device display 122, a combined example of the shade selection and the intensity selection;
a subroutine for requesting, using the one or more processors 250 and the mobile device interface 220, confirmation of the shade selection and the intensity selection after displaying the combined example and before communicating the shade selection and the intensity selection to the color-changing lipstick device;
a subroutine for uploading, using the one or more processors 250, a photograph to a mobile application;
a subroutine for identifying, using the one or more processors 250, a suggested shade option from the plurality of shade options and a suggested intensity option from the plurality of intensity options based upon the photograph;
a subroutine for displaying, using the one or more processors 250 and the mobile device display 122, the suggested shade option and the suggested intensity option;
a subroutine for displaying, using the one or more processors 250 and the mobile device display 122, a combined example of the suggested shade option and the suggested intensity option;
a subroutine for selecting or declining, using the one or more processors 250 and the mobile device display 122, the suggested shade option and the suggested intensity option.
a subroutine for sending, using the one or more processors 250 and the mobile communication system 160, a request for GPS data to the color-changing lipstick device 100;
a subroutine for receiving, using the one or more processors 250 and the mobile communication system 160, sensor data collected by a sensor 164 of the color-changing lipstick device 100;
a subroutine for activating, using the one or more processors 250, an alert of the mobile device 124, for example, wherein the alert includes at least one of displaying, the sensor data on the mobile device display 122, vibrating the mobile device 124, and generating a sound from the mobile device 124;
a subroutine for providing, using the one or more processors 250, a plurality of input options based on the sensor data, wherein the input options may include, for example, adjusting an electrical current provided to a lip product of the color-changing lipstick device, turning on a fan of the color-changing lipstick device, identifying a location of the color-changing lipstick device, beginning a low-power mode of operation of the color-changing lipstick device, selecting a new shade selection from the plurality of shade options, and/or selecting a new intensity selection from the plurality of intensity options;
a subroutine for selecting, using the one or more processors 250 and the mobile device display 122, one or more of the plurality of input options; and
a subroutine for communicating, via the one or more processors 250 and the mobile communication system 160, the selected one or more of the plurality of input options to the color-changing lipstick device.

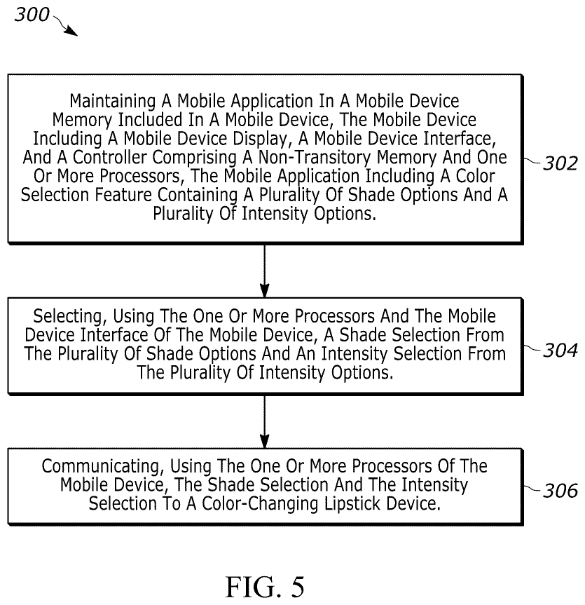

FIG. 5 illustrates schematically a method 300 of controlling an appearance of a lip product 102. At box 302, the method 300 includes maintaining a mobile application in a mobile device memory 248 included in a mobile device 124, the mobile device 124 including a mobile device display 122, a mobile device interface 220, and a controller 200 comprising a non-transitory memory (e.g., program memory 248) and one or more processors 250, the mobile application including a color selection feature containing a plurality of shade options and a plurality of intensity options. At box 304, the method 300 includes selecting, using the one or more processors 250 and the mobile device interface 220 of the mobile device 124, a shade selection from the plurality of shade options and an intensity selection from the plurality of intensity options. At box 306, the method 300 includes communicating, using the one or more processors 250 of the mobile device 124, the shade selection and the intensity selection to a color-changing lipstick device. The method 300 may further include receiving, via a communication module 158 of the color-changing lipstick device 100 and one or more processors 150 of the color-changing lipstick device 100, the selected shape option and the selected intensity option, and adjusting, using one or more processors 150 of the color-changing lipstick device 100, an electrical current provided to a color-changing lip product 102 of the color-changing lipstick device 100 based on the selected shade option and the selected intensity option.

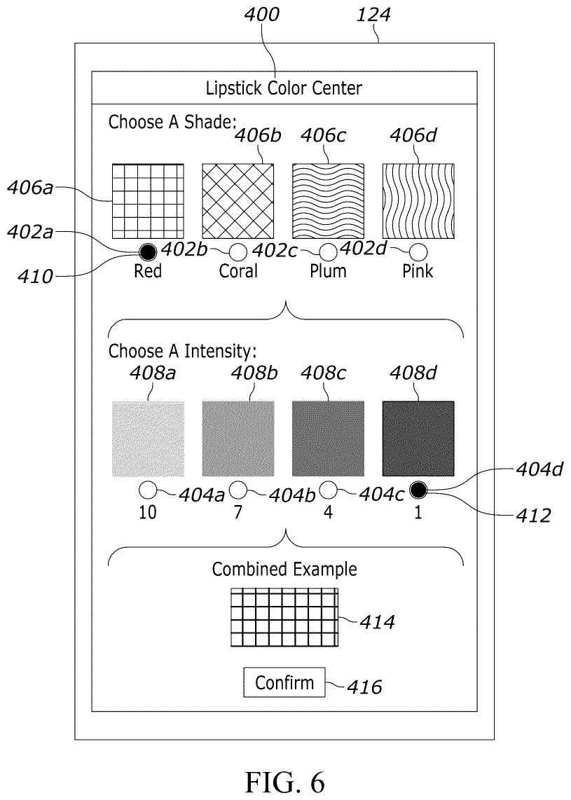

FIG. 6 illustrates a perspective view of a mobile device 124 with a mobile application 400 to control a color-changing lipstick device 100 displaying a plurality of shade options 402 (e.g., 402a, 402b, 402c, 402d) and a plurality of intensity options 404 (e.g., 404a, 404b, 404c, 404d). For example, the plurality of shade options 402 may include red, coral, plum, and pink. The plurality of shade options 402 when implemented by the color-changing lipstick device 100 may correspond with different electrical currents (e.g., a first current for red, a second current for coral, a third current for plum, and a fourth current for pink) that cause the color-changing lip product 102 to attain such shades. The plurality of intensity options may, for example, be a numerical scale from 1 to 10 with 1 being the least intensity (e.g., 1/10^{th} of the electrochromic material 114 activated to change to a selected shade) and 10 being the greatest intensity (e.g., with all of the electrochromic material activated to change to the selected shade). Rather than simply list the shade options and associated currents, the mobile application 400 may display color examples 406 (406a, 406b, 406c, 406d) associated with the plurality of shade options 402 (e.g., pixels on a computer screen may be calibrated to display colors substantially similar to a shade attained by the color-changing lip product 102 when a given shade option is selected). Similarly, the mobile application 400 may also display intensity examples 408 (408a, 408b, 408c, 408d) (e.g., with the brightness of the pixels adjusted to display intensities substantially similar to intensity options 404). When a user makes a shade selection 410 and an intensity selection 412, the mobile application 400 may display a combined example 414 of the shade selection 410 and the intensity selection 412 (e.g., pixels may be calibrated to show a color associated with the selected shade at a brightness associated with a selected intensity). The mobile application may ask for confirmation 416 of a shade selection 410 and the intensity selection 412 after displaying the combined example 414 before communicating the shade selection 410 and intensity selection 412 to the color-changing lipstick device 100.

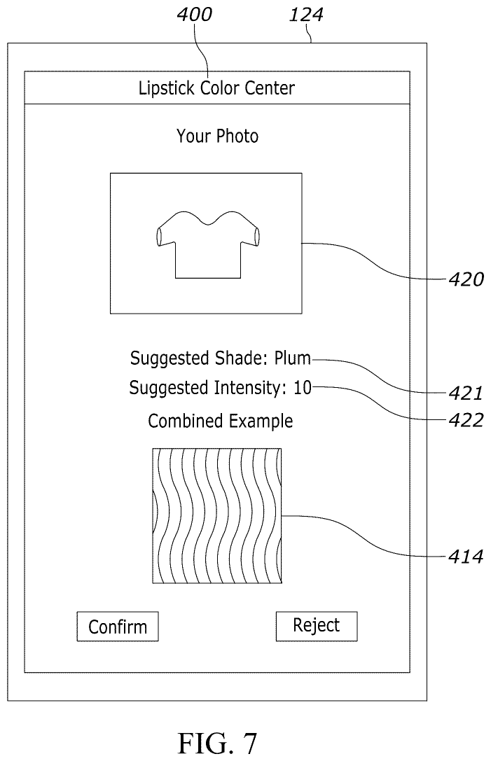

FIG. 7 illustrates a perspective view of the mobile application 400 displaying a photograph-matching feature 418. A photograph 420 may be uploaded to the mobile application 400, and the mobile application 400 will provide a suggested shade option 421 from the plurality of shade options 402 (shown in FIG. 6) and a suggested intensity option 422 from the plurality of intensity options 404) based upon the photograph. For example, if the photograph 420 shows a garment, the mobile application 400 may provide a suggested shade option 421 and a suggested intensity option 422 that matches the shade and intensity of the garment. As another example, if the photograph 420 shows a face, the mobile application 400 may provide a suggested shade option 421 and a suggested intensity option 422 that is considered complimentary to the color of, for example, the cheeks or eyes of the face. The suggested shade option 421 and the suggested intensity option 422 may be displayed separately or as a combined example 414. A user may then select suggested shade option 421 and the suggested intensity option 422 as the shade selection 410 and an intensity selection 412, resulting in implementation by the color-changing lipstick device 100. Alternately, the user may decline the suggested shade option 421 and the suggested intensity option 422. The mobile application 400 may then provide alternate suggestions based upon, for example, the same photograph 420 (e.g., after selection of a particular focus area by a user) or a new photograph 420.

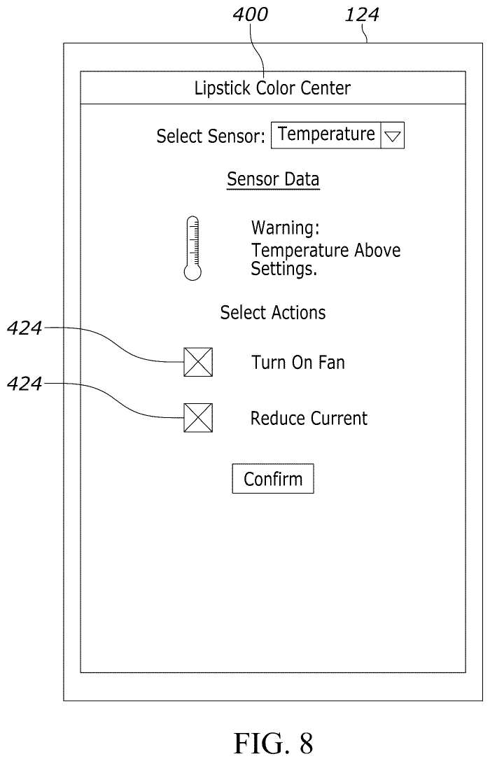

FIG. 8 illustrates a perspective view of the mobile application 400 displaying the functionality of the mobile application 400 in association with one or more sensors 164. The sensors 164 may include any of a temperature sensor, a GPS sensor, a UV sensor, a battery sensor, a light sensor, and a motion sensor. The mobile application 400 can send a request for sensor data from a sensor 164, such as a GPS sensor. The mobile application 400 can also receive, in response to the request or unprompted, sensor data from a sensor 164. In response, the mobile application 400 can offer a variety of options. For example, the mobile application may activate an alert of the mobile device (e.g., using a speaker, vibration device, or other feedback mechanism of the mobile device) or may display the sensor data on the display 122 of the mobile device 124.

In response to the sensor data, the mobile application may, as shown in FIG. 8, provide a plurality of input options 424. A user may select one or more of the plurality of input options 424, and the selection may then be communicated to the color-changing lipstick device 100. The input options 424 may include, for example, adjusting an electrical current provided to a lip product 102 of the color-changing lipstick device 100, turning on a fan of the color-changing lipstick device 100, identifying a location of the color-changing lipstick device 100, beginning a low-power mode of operation of the color-changing lipstick device 100, selecting a new shade selection from the plurality of shade options 402, and selecting a new intensity selection from the plurality of intensity options 404.

The color-changing lipstick device 100 may receive, via the communication module 158 and the controller 118, the selected one or more of the plurality of input options 424 and may then implement the selected one or more of the plurality of input options 424. For example, when the selected one or more of the plurality of input options 424 includes at least one of adjusting an electrical current provided to the lip product 102 of the color-changing lipstick device 100, selecting a new shade selection 410 from the plurality of shade options 402, and selecting a new intensity selection 412 from the plurality of intensity options 404, implementing the selected one or more of the plurality of input options 424 includes controlling a power source 116 using the controller 118. When the selected one or more of the plurality of input options 424 includes turning on a fan 173 of the color-changing lipstick device 100, implementing the selected one or more of the plurality of input options 424 includes controlling the fan 173 using the controller 118. When the selected one or more of the plurality of input options 424 includes identifying a location of the color-changing lipstick device 100, implementing the selected one or more of the plurality of input options 424 includes activating, using the controller 118, a GPS sensor 164 to collect sensor data regarding a location of the color-changing lipstick device 100, and communicating, using the communication module 158 and the controller 118, the sensor data regarding the location of the color-changing lipstick device 100 to the mobile device. When the selected one or more of the plurality of input options 424 includes beginning a low-power mode of operation of the color-changing lipstick device 100, implementing the selected one or more of the plurality of input options 424 includes controlling the power source 116 using the controller 118 to reduce usage of the power source 116.

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” or “some embodiments” means that a particular element, feature, structure, or characteristic described in connection with the embodiment is included in at least one embodiment. The appearances of the phrase “in one embodiment” or “in some embodiments” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of “a” or “an” is employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for cosmetic application, and/or systems, methods, and/or techniques associated therewith. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A method of controlling an appearance of a lip product, the method comprising:
maintaining a mobile application in a mobile device memory included in a mobile device, the mobile device including a mobile device display, a mobile device interface, and a controller comprising a non-transitory memory and one or more processors, the mobile application including a color selection feature containing a plurality of shade options and a plurality of intensity options;
selecting, using the one or more processors and the mobile device interface of the mobile device, a shade selection from the plurality of shade options and an intensity selection from the plurality of intensity options; and
communicating, using the one or more processors of the mobile device, the shade selection and the intensity selection to a color-changing lipstick device.

2. The method of claim 1, the method further comprising:
uploading, using the one or more processors of the mobile device, a photograph to the mobile application;
identifying, using the one or more processors of the mobile device, a suggested shade option from the plurality of shade options and a suggested intensity option from the plurality of intensity options based upon the photograph; and
displaying, using the one or more processors of the mobile device and the mobile device display, the suggested shade option and the suggested intensity option.

3. The method of claim 2, the method further comprising:
displaying, using the one or more processors of the mobile device and the mobile device display, a combined example of the suggested shade option and the suggested intensity option.

4. The method of claim 2, the method further comprising:
selecting or declining, using the one or more processors of the mobile device and the mobile device interface, the suggested shade option and the suggested intensity option.

5. The method of claim 1, the method further comprising sending, using the one or more processors of the mobile device, a request for GPS data to the color-changing lipstick device.

6. The method of claim 1, the method further comprising receiving, via a communication module of the color-changing lipstick device and the one or more processors of the color-changing lipstick device, the shade selection and the intensity selection, and adjusting, using the one or more processors of the color-changing lipstick device, an electrical current provided to a color-changing lip product of the color-changing lipstick device based on the shade selection and the intensity selection.

7. The method of claim 1, the method further comprising:
receiving, using the one or more processors of the mobile device, sensor data collected by a sensor of the color-changing lipstick device; and
activating, using the one or more processors, an alert of the mobile device.

8. The method of claim 7, wherein the alert includes displaying, using the one or more processors of the mobile device, the sensor data on the mobile device display.

9. The method of claim 7, wherein the alert includes vibrating, using the one or more processors of the mobile device, the mobile device.

10. The method of claim 7, wherein the alert includes generating, using the one or more processors of the mobile device, a sound from the mobile device.

11. The method of claim 7 further comprising:
providing, using the one or more processors of the mobile device, a plurality of input options based on the sensor data;
selecting, using the one or more processors of the mobile device and the mobile device interface, one or more of the plurality of input options; and
communicating, via the one or more processors of the mobile device, the selected one or more of the plurality of input options to the color-changing lipstick device.

12. The method of claim 7, wherein the sensor data includes sensor data from at least one of a temperature sensor, a GPS sensor, a UV sensor, a battery sensor, a light sensor, and a motion sensor.

13. The method of claim 11, wherein the input options include adjusting an electrical current provided to the lip product of the color-changing lipstick device, turning on a fan of the color-changing lipstick device, identifying a location of the color-changing lipstick device, beginning a low-power mode of operation of the color-changing lipstick device, selecting a new shade selection from the plurality of shade options, and selecting a new intensity selection from the plurality of intensity options.

14. The method of claim 11, the method further comprising receiving, via a communication module of the color-changing lipstick device and the one or more processors of the color-changing lipstick device, the selected one or more of the plurality of input options and implementing the selected one or more of the plurality of input options.

15. The method of claim 14, wherein the selected one or more of the plurality of input options includes at least one of adjusting an electrical current provided to the lip product of the color-changing lipstick device, selecting a new shade selection from the plurality of shade options, and selecting a new intensity selection from the plurality of intensity options, and
implementing the selected one or more of the plurality of input options includes controlling a power source, using the one or more processors of the color-changing lipstick device, to adjust the electrical current provided to the lip product.

16. The method of claim 14, wherein the selected one or more of the plurality of input options includes turning on a fan of the color-changing lipstick device, and implementing the selected one or more of the plurality of input options includes controlling the fan, using the one or more processors of the color-changing lipstick device.

17. The method of claim 14, wherein the selected one or more of the plurality of input options includes identifying a location of the color-changing lipstick device, and implementing the selected one or more of the plurality of input options includes activating, using the one or more processors of the color-changing lipstick device, a GPS sensor to collect sensor data regarding a location of the color-changing lipstick device, and communicating, using the communication module of the color-changing lipstick device and the one or more processors of the color-changing lipstick device, the sensor data regarding the location of the color-changing lipstick device to the mobile device.

18. The method of claim 14, wherein the selected one or more of the plurality of input options includes beginning a low-power mode of operation of the color-changing lipstick device, and implementing the selected one or more of the plurality of input options includes controlling a power source, using the one or more processors of the color-changing lipstick device, to reduce usage of the power source.

19. The method of claim 1, wherein the method further includes displaying, using the one or more processors of the mobile device and the mobile device display, color examples in conjunction with the plurality of shade options.

20. The method of claim 1, wherein the method further includes displaying, using the one or more processors of the mobile device and the mobile device display, intensity examples in conjunction with the plurality of intensity options.

21. The method of claim 1, wherein the method further includes displaying, using the one or more processors of the mobile device and the mobile device display, a combined example of the shade selection and the intensity selection.

22. The method of claim 23, wherein the method further includes requesting, using the one or more processors of the mobile device and the mobile device interface, confirmation of the shade selection and the intensity selection after displaying the combined example and before communicating the shade selection and the intensity selection to the color-changing lipstick device.
